(19) United States
(12) Patent Application Publication
(10) Pub. No.: US 2010/0234781 A1
(43) Pub. Date: Sep. 16, 2010

(54) Cast protector

(76) Inventor: Peter Cassidy, Winter Haven, FL (US)

Correspondence Address: Pennington, Moore, Wilkinson, Bell & Dunbar, P.A., Post Office Box 10095, 215 S. Monroe Street, 2nd Floor, Tallahassee, FL, 32302-2095, US

(21) Appl. No.: 12/381,253

(22) Filed: Mar. 10, 2009

(51) Int. Cl. A61F 5/00

(52) U.S. Cl. 602/3

(57) ABSTRACT

The present invention comprises a cover for enclosing a cast on a user's arm and/or a user's leg. The cover is generally comprised of a sheath and a first ring. The sheath has a first end and a second end. Proximate to the first end of the sheath is a finger sheath and a thumb sheath. The second end of the sheath has a first ring configured to frictionally engage the arm of the user and a first opening. The sheath is rolled around the first ring for easily putting on or taking off the cover.

### CROSS-REFERENCES TO RELATED APPLICATIONS

Not Applicable

### STATEMENT REGARDING FEDERALLY SPONSORED RESEARCH OR DEVELOPMENT

Not Applicable

### MICROFICHE APPENDIX

Not Applicable

### BACKGROUND OF THE INVENTION

1. Field of the Invention

This invention relates to the field of devices made for covering and protecting arm or leg casts. More specifically, the invention comprises a glove which extends over a user's arm cast while still allowing use of the user's hand.

2. Description of the Related Art

Orthopedic casts are used to heal a broken bone by holding the bone in place until fully healed. It is important to keep the cast and area around the cast dry and free from contaminants, including sand or particles of dirt, during the time in which the cast is worn.

Previously to protect an orthopedic cast a plastic bag wrapped with tape could be used to keep the cast dry. However, it is difficult to achieve an effective seal around the cast using plastic bags and tape. Additionally, the plastic bag does not enable the user to grasp objects easily.

Therefore what is needed is a cast covering, allowing the user to grasp objects, which is quick and easy to put on while keeping an effective seal to keep the cast dry and free of contaminants. The present invention achieves this objective, as well as others that are explained in the following description.

### BRIEF SUMMARY OF THE INVENTION

The present invention comprises a cover for enclosing a cast on a user's arm or leg. The cover is generally comprised of a sheath, a finger sheath, a thumb sheath and a first ring. The sheath has a first end and a second end. Proximate to the first end of the sheath is the finger sheath and thumb sheath which allow a user to grasp objects while using the present cover. The second end of the sheath has a first ring configured to frictionally engage the arm of the user and a first opening. The sheath can be rolled around the first ring for easily rolling on or taking off the cover.

The cover is easy to put on, keeps the user's cast dry and clean, while allowing the user to still be able to easily grasp objects.

### BRIEF DESCRIPTION OF THE SEVERAL VIEWS OF THE DRAWINGS

FIG. 1 is a perspective view, showing the preferred embodiment of the present invention.

FIG. 2 is a perspective view, showing the preferred embodiment of the present invention.

FIG. 3 is a perspective cut away view, showing the present invention covering a user's arm.

FIG. 4 is a perspective view, showing the present invention.

FIG. 5 is a perspective view, showing the present invention in a compact state.

FIG. 6 is a perspective view, showing the present invention being placed over a user's hand.

FIG. 7 is a perspective view, showing the present invention being placed over a user's arm.

FIG. 8 is a cross-section view, showing the present invention in a compact state.

FIG. 9 is a perspective view, showing the user of the present invention grasping a bottle.

FIG. 10 is a perspective view, showing an alternate embodiment of the present invention.

FIG. 11 is a perspective view, showing an alternate embodiment of the present invention.

FIG. 12 is a perspective view, showing an alternate embodiment of the present invention.

FIG. 13 is a perspective view, showing an alternate embodiment of the present invention.

### REFERENCE NUMERALS IN THE DRAWINGS

| | | | |
|---|---|---|---|
| 10 | arm | 12 | cast |
| 14 | first ring | 16 | sheath |
| 18 | finger sheath | 20 | fingers |
| 22 | thumb | 24 | thumb sheath |
| 26 | inside wall | 28 | bottle |
| 30 | second ring | 32 | third ring |
| 34 | first opening | 36 | second opening |
| 38 | third opening | 40 | thumb sheath |
| 42 | index finger sheath | 44 | middle finger sheath |
| 46 | ring finger sheath | 48 | little finger sheath |
| 50 | present cover | 52 | leg ring |
| 54 | leg sheath | 56 | reinforced sole |
| 58 | leg | | |

### DETAILED DESCRIPTION OF THE INVENTION

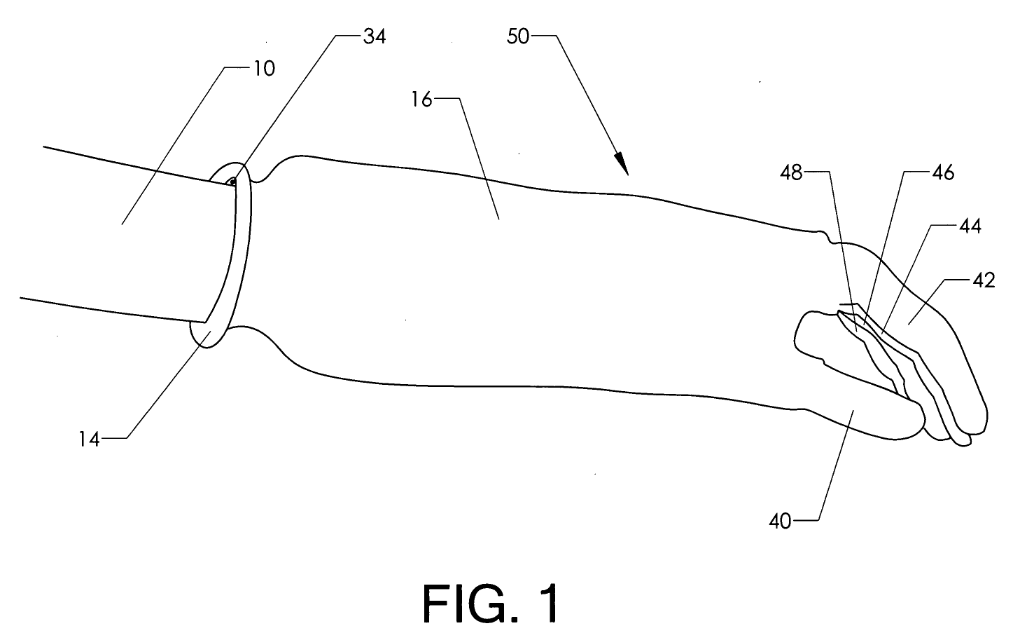

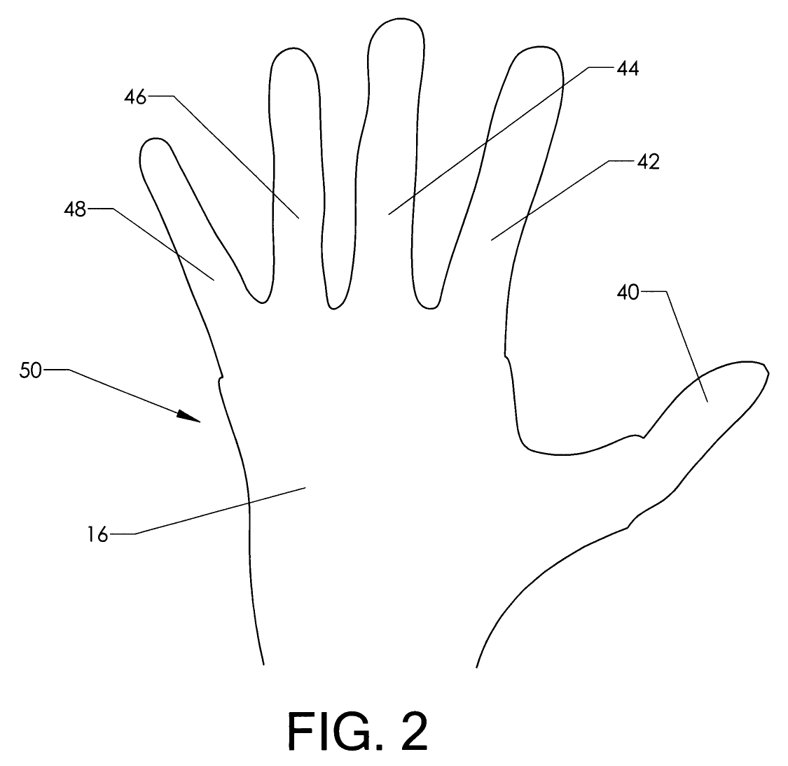

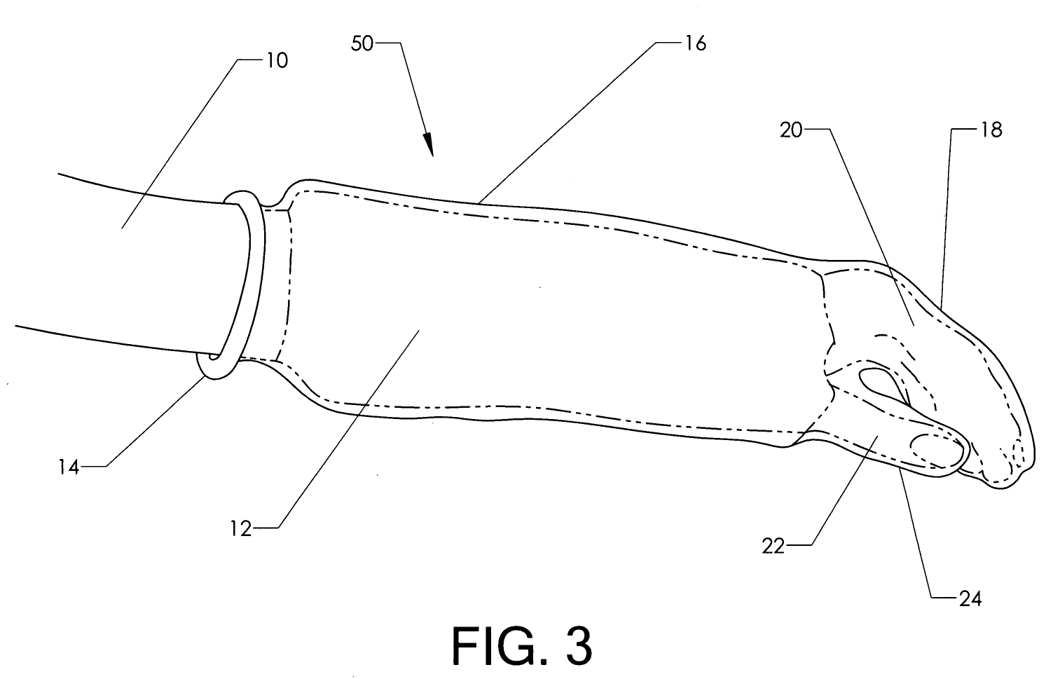

FIG. 1 and FIG. 2 illustrate the present invention in the preferred embodiment. The present cover 50 covers a user's cast 12 (shown in FIG. 3) and prevents a user's cast 12 (shown in FIG. 3) from getting wet or dirty. The present cover 50 is generally made up of a sheath 16, thumb sheath 40, index finger sheath 42, middle finger sheath 44, ring finger sheath 46, little finger sheath 48, first ring 14 and first opening 34. User's fingers fit into each finger sheath, the user's index finger fits into index finger sheath 42, the user's middle finger fits into middle finger sheath 44, the user's ring finger fits into ring finger sheath 46 and the user's little finger fits into little finger sheath 48. The present embodiment of the present cover 50 allows full use of each of the user's finger. As shown in FIG. 1 sheath 16 covers the length of the cast and first ring 14 creates a seal between the present cover 50 and the user's arm 10.

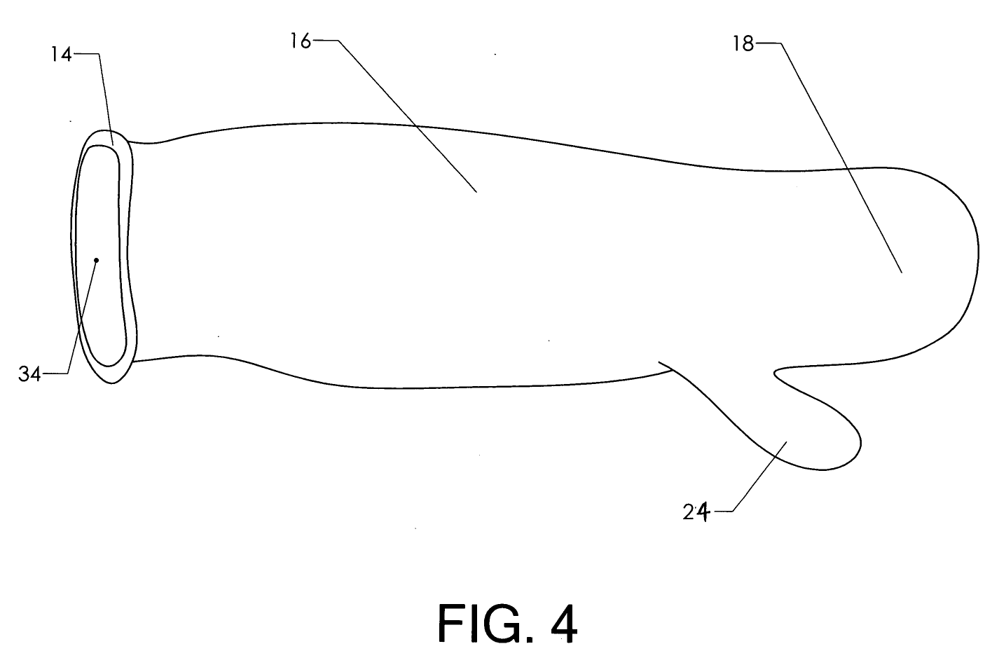

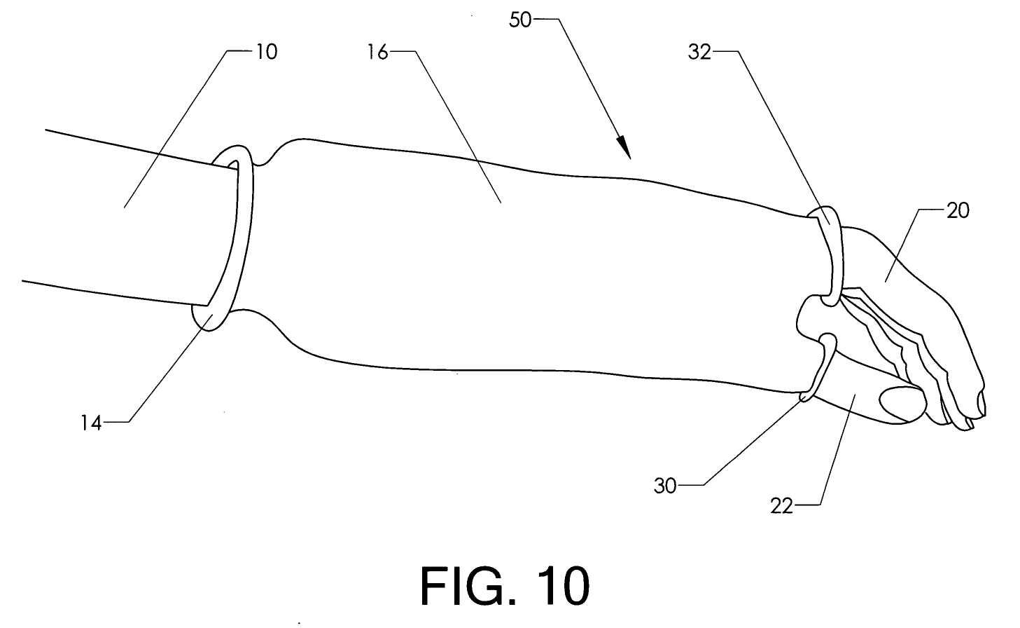

FIG. 3 illustrates the present invention in a similar embodiment in which each individual finger sheath is combined into one finger sheath 18 which covers all of user's fingers, similar to a mitten. The present cover 50 covers a user's cast 12, shown here in broken lines. Thumb sheath 24 and finger sheath 18 are located at the end of sheath 16, configured to fit over the user's hand. While both thumb sheath 24 and finger sheath 18 are shown as enclosed sheaths, the sheaths do not have to be enclosed, as shown in FIG. 10-12. Returning to FIG. 3, first ring 14 is located at the opposite end of sheath 16 and is configured to frictionally engage user's arm 10. First ring 14 has a diameter in a relaxed state that is less than the diameter of sheath 16 in a relaxed state, thereby allowing sheath 16 to fully expand to cover cast 12 while first ring 14 fits closely to user's arm 10 to prevent water or dirt from entering first opening 34, shown in FIG. 4. Returning to FIG. 3, in the present embodiment thumb sheath 24 covers thumb 22 while finger sheath 18 covers fingers 20. Cast 12, which incorporates thumb joint, can be fully enclosed by thumb sheath 24. Sheath 16 covers cast 12 shielding cast 12 from the elements, such as water or dirt.

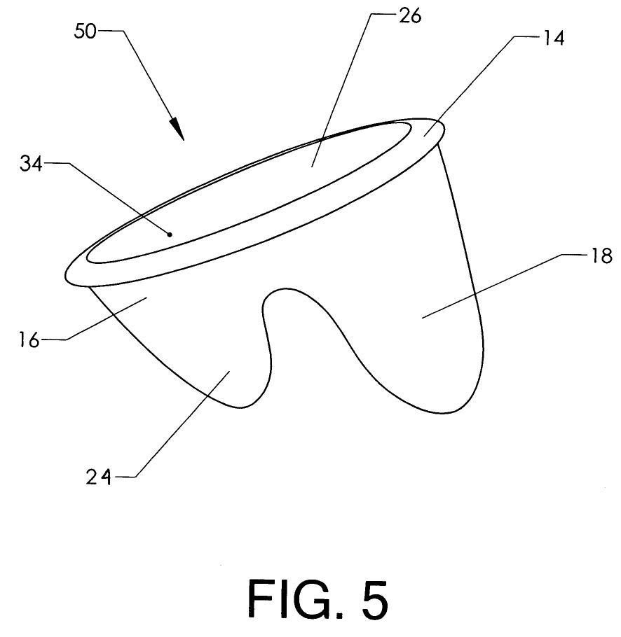

FIG. 5 is a perspective view showing cover 50 in a compact state. FIG. 5 shows sheath 16 rolled around first ring 14. Rolling sheath 16 around first ring 14 makes cover 50 easier to store and easier to apply to user's cast 12, as described below. Cover 50 is preferably elastomeric and made from a material such as latex or polyurethane. Additionally, inside wall 26 and exterior of cover 50 are preferably coated with a powder, such as talcum powder, to enable cover 50 to be taken on and off easily.

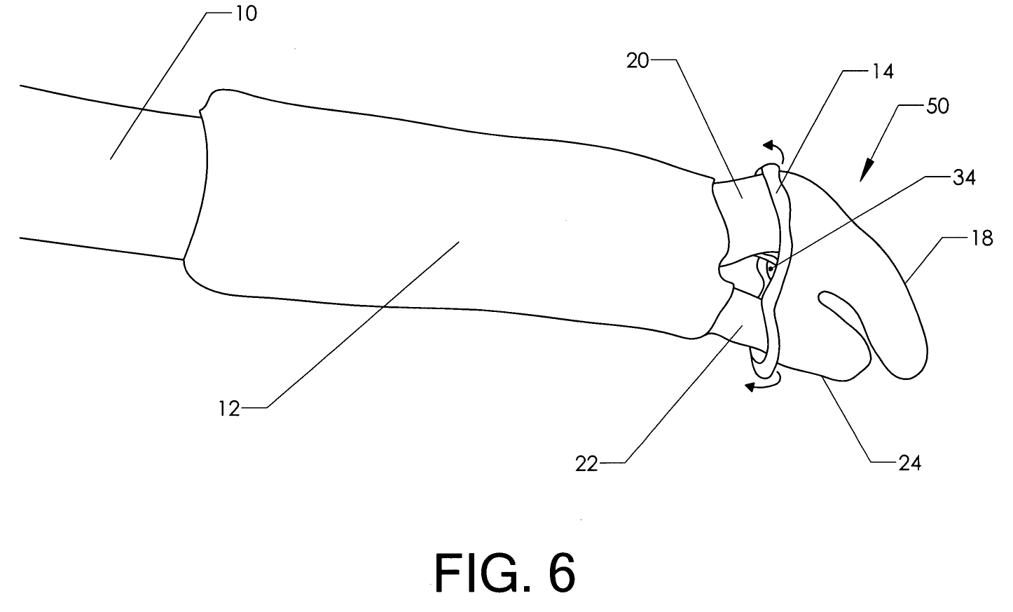

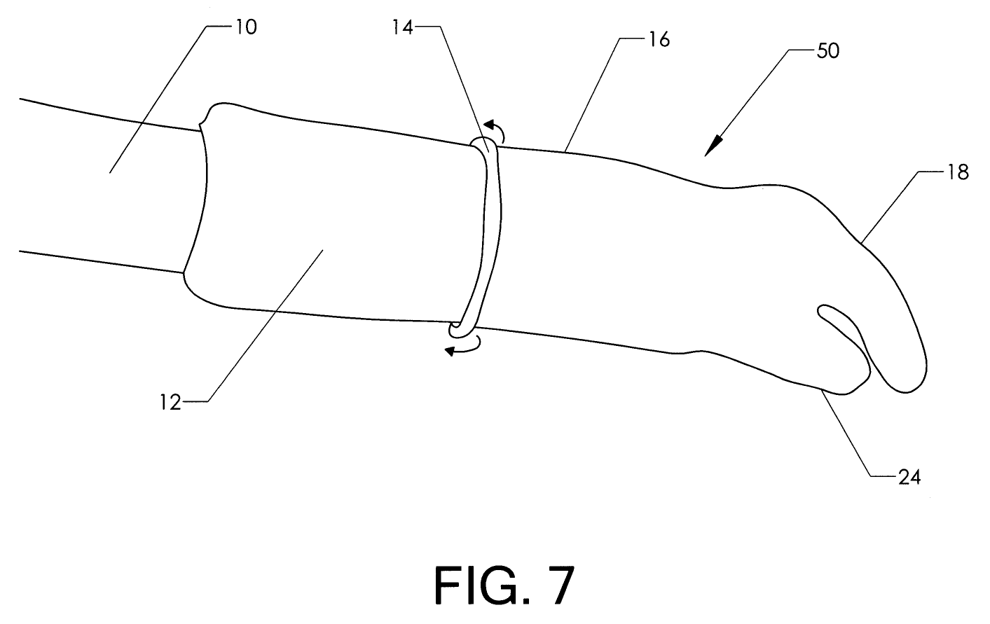

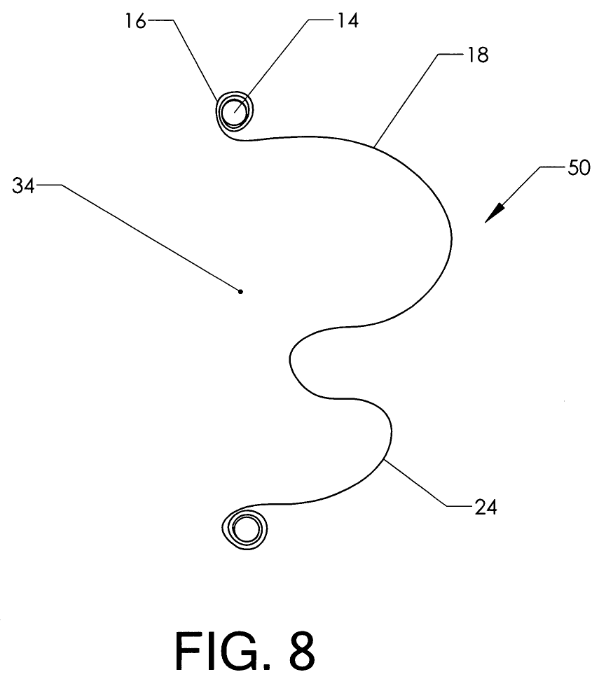

First opening 34 accepts user's hand as shown in FIG. 6. User's fingers 20 are placed in finger sheath 18 while user's thumb 22 fits into thumb sheath 24. First ring 14 rolls along user's fingers 20 and thumb 22 towards cast 12. As first ring 14 is unrolled over cast 12 sheath 16 is released from first ring 14 covering cast as shown in FIG. 7. The function of releasing sheath 16 by unrolling first ring 14 along user's cast 12 prevents the user from having to pull and fight with sheath 16 to fit it over cast 12. Any friction between cast 12 and inside wall 26 (shown in FIG. 7) of sheath 16 is eliminated by the rolling function as each point on inside wall 26 of sheath 16 contacts cast 12 at only one point along cast 12 and remains at that location until the roll function is reversed and cover 50 is taken off of user's arm 10. A cross-section of the present cover 50 is shown in FIG. 8. Sheath 16 is shown rolled up around first ring 14.

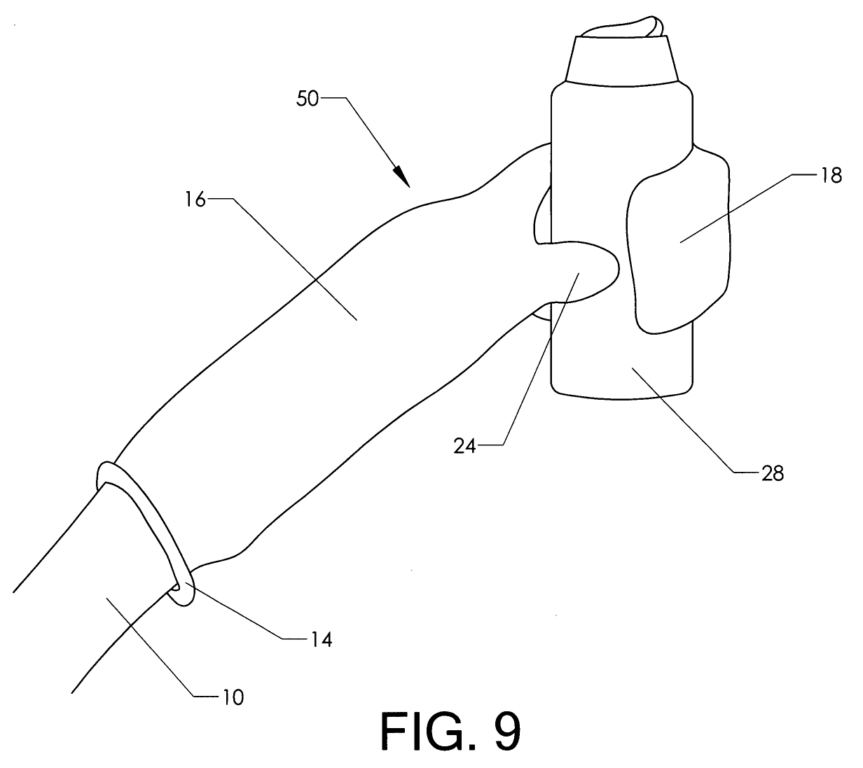

FIG. 9 illustrates the user grasping a bottle 28 while wearing the present cover 50. The location of finger sheath 18 and thumb sheath 24 on the present cover 50 enable the user to grasp objects such as a bottle 28. The user's ability to grasp objects while wearing the present cover 50 helps the user maintain functionality of the user's hand while still keeping the cast free from dirt or water.

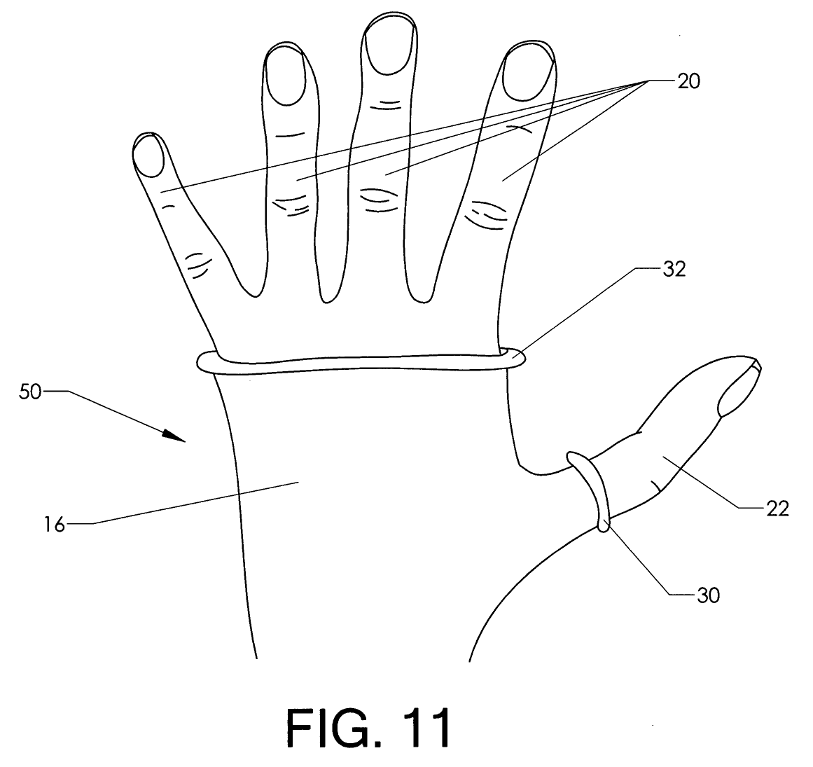

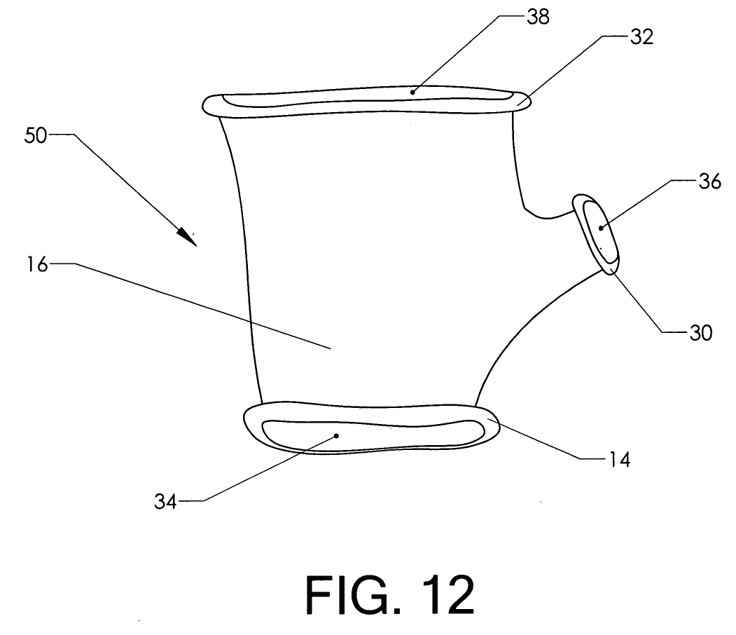

FIG. 10 shows an alternate embodiment of the present invention. Alternate embodiment of present cover 50 allows user's fingers 20 and user's thumb 22 to be exposed while still protecting the user's cast 12 (not shown) from liquid, dirt or other contaminants. As shown in FIG. 1, second ring 30 fits onto user's thumb 22 while third ring 32 fits just below user's fingers 20. Second ring 30 and third ring 32 fit snugly against user preventing contaminants from accessing user's cast 12 (not shown). Alternate embodiment of present cover 50 is shown apart from user in FIG. 12. User's thumb 22 (shown in FIG. 11) fits into second opening 36 while user's fingers 20 (shown in FIG. 11) fit into third opening 38.

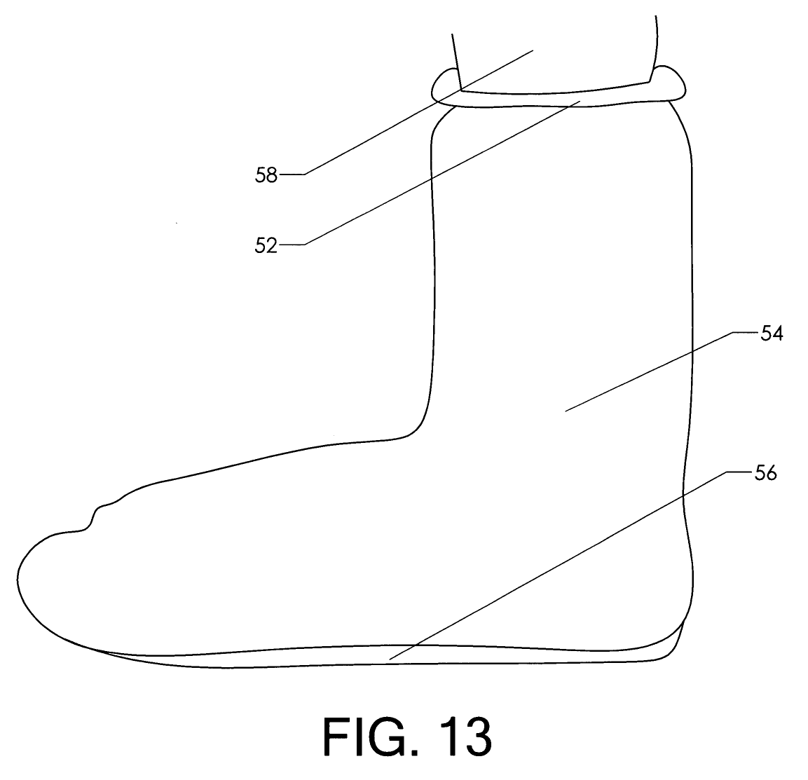

As illustrated in FIG. 13 shows an alternate embodiment of the present invention. In this embodiment leg sheath 54 encloses the toes and foot of user and extends over the user's cast. Leg ring 52 engages with leg 58 on top of the cast creating a seal against leg 58 so that water and/or debris cannot enter the user's cast. Leg ring 52 can fit below user's knee or above user's knee according to the size of user's leg cast. The leg ring 52, shown in this figure, functions in the same way as the first ring functions, described above. Additionally, leg sheath 54 contains a reinforced sole 56 which is located on the bottom of the user's foot. Reinforced sole 56 cushions the user's foot as the user walks and provides added protection from tearing. In the preferred embodiment leg sheath 54 will be made up of latex or polyurethane. In order to manufacture the present device dip molding will likely be used. Dip molding consists of dipping a mold into a desired solution until the wall thickness is sufficiently built up. It is in this way that reinforced sole 56 can be easily manufactured. Reinforced sole 56 would be dipped into the desired solution more times or for a longer period of time than the rest of the device until the desired thickness is reached on the bottom of the device.

The preceding description contains significant detail regarding the novel aspects of the present invention. It should not be construed, however, as limiting the scope of the invention but rather as providing illustrations of the preferred embodiments of the invention. As an example, the present cover 50 could be made up from any number of materials. Additionally, present cover 50 can be glove-like or mitten-like. Thus, the scope of the invention should be fixed by the following claims, rather than by the examples given.

Having described my invention, I claim:

1. A cover which encloses a user's cast on an arm of said user, said user having fingers and a thumb, said cover comprising:
a. a sheath, having a first end and a second end:
i. wherein said sheath has a diameter in a relaxed state;
ii. wherein said sheath has a first opening at said second end;
iii. wherein said sheath has an exterior wall and an interior wall;
b. a finger sheath, wherein said finger sheath is located at said first end of said sheath;
c. a thumb sheath, wherein said thumb sheath is located at said first end of said sheath proximate to said finger sheath;
d. a first ring:
i. wherein said first ring has a diameter in a relaxed state;
ii. wherein said first ring is configured to frictionally engage said arm of said user above said cast;
iii. wherein said diameter of said first ring in said relaxed state expands less than said diameter of said sheath in said relaxed state; and
iv. wherein said first ring is configured to roll said sheath around said first ring.

2. A cover as recited in claim 1, wherein said cover is elastomeric.

3. A cover as recited in claim 1, wherein said cover is comprised of latex.

4. A cover as recited in claim 2, wherein said cover is coated with talcum powder.

5. A cover as recited in claim 1, wherein said finger sheath further comprises an index finger sheath, a middle finger sheath, a ring finger sheath and a little finger sheath.

6. A cover as recited in claim 1, wherein said first end of said sheath further comprises:
i. a second opening at said thumb sheath; and
ii. wherein said second opening is defined by a second ring.

7. A cover as recited in claim 6, wherein said first end of said sheath further comprises:
i. a third opening at said finger sheath; and
ii. wherein said third opening is defined by a third ring.

8. A cover as recited in claim 1, wherein said first end of said sheath further comprises an index finger sheath, a middle finger sheath, a ring finger sheath and a little finger sheath configured to individually enclose said fingers of said user.

9. A cover as recited in claim 1, wherein said cover is scented.

10. A cover which encloses a user's cast on an arm of said user, said user having a plurality of fingers and a thumb, said cover comprising:
a. a sheath, having a first end and a second end:
i. wherein said sheath encloses said fingers at said first end;
ii. wherein said sheath encloses said thumb at said first end;
iii. wherein said sheath has a first opening at said second end;
iv. wherein said sheath has a diameter in a relaxed state;
v. wherein said sheath has an exterior wall and an interior wall;
b. a first ring:
i. wherein said first ring has a diameter in a relaxed state;
ii. wherein said first ring is configured to frictionally engage said arm of said user above said cast;
iii. wherein said diameter of said first ring in said relaxed state expands less than said diameter of said sheath in said relaxed state; and
iv. wherein said first ring is configured to roll said sheath around said first ring such that said sheath is wrapped around said first ring.

11. A cover as recited in claim 10, wherein said cover is elastomeric.

12. A cover as recited in claim 10, wherein said sheath encloses each of said plurality of fingers such that user can operate each of said plurality of fingers independently.

13. A cover as recited in claim 11, wherein said cover is coated with talcum powder.

14. A cover as recited in claim 11, wherein said cover is comprised of latex.

15. A cover as recited in claim 10, wherein said cover is opaque.

16. A cover as recited in claim 10, wherein said cover is scented.

17. A cover which encloses a user's cast on a leg and a foot of said user, said foot of said user having a top and a bottom, said cover comprising:
a. a sheath, having a first end and a second end:
i. wherein said sheath is enclosed at said first end around said foot of said user;
ii. wherein said sheath has a first opening at said second end;
iii. wherein said sheath has a diameter in a relaxed state;
iv. wherein said sheath has an exterior wall and an interior wall;
b. a first ring:
i. wherein said first ring surrounds said first opening;
ii. wherein said first ring has a diameter in a relaxed state;
iii. wherein said first ring is configured to frictionally engage said leg of said user above said cast;
iv. wherein said diameter of said first ring in said relaxed state expands less than said diameter of said sheath in said relaxed state; and
v. wherein said first ring is configured to roll said sheath around said first ring such that said sheath is wrapped around said first ring;
c. a reinforced sole:
i. wherein said reinforced sole is integrated with said sheath such that said reinforced bottom sits along said bottom of said user's foot.

18. A cover as recited in claim 17, wherein said cover is elastomeric.

19. A cover as recited in claim 17, wherein said cover is coated with talcum powder.

20. A cover as recited in claim 17, wherein said cover is comprised of latex.
